SEQUENCE LISTING (40 sequences)

SEQ ID NO: 1 - DNA, 12 bp, Artificial Sequence
  synthetic
tctagagccaca

SEQ ID NO: 2 - DNA, 396 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgc

SEQ ID NO: 3 - DNA, 54 bp, Artificial Sequence
  synthetic
ggttctacaagtgggagcggaaagccaggatctggcgaaggtagcaccaaaggc

SEQ ID NO: 4 - DNA, 354 bp, Artificial Sequence
  synthetic
caggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctca

SEQ ID NO: 5 - DNA, 45 bp, Artificial Sequence
  synthetic
agatctgaacaaaaacttatatctgaggaagacctggggtcgacg

SEQ ID NO: 6 - DNA, 144 bp, Artificial Sequence
  synthetic
accacaacgccagctccccgcccaccaacgcctgcgccaactattgcctcacagcctttgagtctccggccagaagcatgtcgccccgctgccggtggagcagtccatacaagaggccttgacttcgcgtgcgatatctcgagc

SEQ ID NO: 7 - DNA, 81 bp, Artificial Sequence
  synthetic
ttctgggtgttggtcgttgtgggtggtgtcctggcgtgttattcactgttggttactgtggcttttataattttctgggtg

SEQ ID NO: 8 - DNA, 186 bp, Artificial Sequence
  synthetic
aaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacag

SEQ ID NO: 9 - DNA, 123 bp, Artificial Sequence
  synthetic
aggtctcaagttagaaaagcagctataacatcttatgagaaatctgatggagtatatacagggctcagcacgcgaaatcaggagacctatgaaactctgaagcatgagaagcccccgcagtag

SEQ ID NO: 10 - DNA, 17 bp, Artificial Sequence
  synthetic
ggcggccgcgaattcgc

SEQ ID NO: 11 - protein, 132 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKR

SEQ ID NO: 12 - protein, 18 aa, Artificial Sequence
  synthetic
GSTSGSGKPGSGEGSTKG

SEQ ID NO: 13 - protein, 118 aa, Artificial Sequence
  synthetic
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSS

SEQ ID NO: 14 - protein, 15 aa, Artificial Sequence
  synthetic
RSEQKLISEEDLGST

SEQ ID NO: 15 - protein, 48 aa, Artificial Sequence
  synthetic
TTTPAPRPPTPAPTIASQPLSLRPEACRPAAGGAVHTRGLDFACDISS

SEQ ID NO: 16 - protein, 27 aa, Artificial Sequence
  synthetic
FWVLVVVGGVLACYSLLVTVAFIIFWV

SEQ ID NO: 17 - protein, 62 aa, Artificial Sequence
  synthetic
KKVAKKPTNKAPHPKQEPQEINFPDDLPGSNTAAPVQETLHGCQPVTQEDGKESRISVQERQ

SEQ ID NO: 18 - protein, 40 aa, Artificial Sequence
  synthetic
RSQVRKAAITSYEKSDGVYTGLSTRNQETYETLKHEKPPQ

SEQ ID NO: 19 - DNA, 123 bp, Artificial Sequence
  synthetic
cgatctaaaagaagcaggctcttgcactccgattacatgaatatgaccccgagacggcccggacctacgcggaaacattaccaaccttatgcaccaccgagggacttcgctgcttatcgctcc

SEQ ID NO: 20 - protein, 41 aa, Artificial Sequence
  synthetic
RSKRSRLLHSDYMNMTPRRPGPTRKHYQPYAPPRDFAAYRS

SEQ ID NO: 21 - DNA, 66 bp, Artificial Sequence
  synthetic
gccctggtggtgatccccatcatcttcggaatcctgtttgccatcctcttggtgctggtctttatc

SEQ ID NO: 22 - protein, 22 aa, Artificial Sequence
  synthetic
ALVVIPIIFGILFAILLVLVFI

SEQ ID NO: 23 - DNA, 126 bp, Artificial Sequence
  synthetic
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 24 - protein, 42 aa, Artificial Sequence
  synthetic
KRGRKKLLYIFKQPFMRPVQTTQEEDGCSCRFPEEEEGGCEL

SEQ ID NO: 25 - DNA, 1368 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcgccctggtggtgatccccatcatcttcggaatcctgtttgccatcctcttggtgctggtctttatcaaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacagaggtctcaagttagaaaagcagctataacatcttatgagaaatctgatggagtatatacagggctcagcacgcgaaatcaggagacctatgaaactctgaagcatgagaagcccccgcagtag

SEQ ID NO: 26 - protein, 455 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSALVVIPIIFGILFAILLVLVFIKKVAKKPTNKAPHPKQEPQEINFPDDLPGSNTAAPVQETLHGCQPVTQEDGKESRISVQERQRSQVRKAAITSYEKSDGVYTGLSTRNQETYETLKHEKPPQ

SEQ ID NO: 27 - DNA, 1329 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgcgatctaaaagaagcaggctcttgcactccgattacatgaatatgaccccgagacggcccggacctacgcggaaacattaccaaccttatgcaccaccgagggacttcgctgcttatcgctccggaggtggcaggtctcaagttagaaaagcagctataacatcttatgagaaatctgatggagtatatacagggctcagcacgcgaaatcaggagacctatgaaactctgaagcatgagaagcccccgcagtag

SEQ ID NO: 28 - protein, 442 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVRSKRSRLLHSDYMNMTPRRPGPTRKHYQPYAPPRDFAAYRSGGGRSQVRKAAITSYEKSDGVYTGLSTRNQETYETLKHEKPPQ

SEQ ID NO: 29 - DNA, 1383 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgaaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacagaggtctcaagttagaaaagcagctataacatcttatgagaaatctgatggagtatatacagggctcagcacgcgaaatcaggagacctatgaaactctgaagcatgagaagcccccgcagtag

SEQ ID NO: 30 - protein, 460 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVKKVAKKPTNKAPHPKQEPQEINFPDDLPGSNTAAPVQETLHGCQPVTQEDGKESRISVQERQRSQVRKAAITSYEKSDGVYTGLSTRNQETYETLKHEKPPQ

SEQ ID NO: 31 - DNA, 1323 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgaagcgcggtcggaagaagctgctgtacatctttaagcaacccttcatgaggcctgtgcagactactcaagaggaggacggctgttcatgccggttcccagaggaggaggaaggcggctgcgaactgaggtctcaagttagaaaagcagctataacatcttatgagaaatctgatggagtatatacagggctcagcacgcgaaatcaggagacctatgaaactctgaagcatgagaagcccccgcagtag

SEQ ID NO: 32 - protein, 440 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVKRGRKKLLYIFKQPFMRPVQTTQEEDGCSCRFPEEEEGGCELRSQVRKAAITSYEKSDGVYTGLSTRNQETYETLKHEKPPQ

SEQ ID NO: 33 - DNA, 1455 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgcgatctaaaagaagcaggctcttgcactccgattacatgaatatgaccccgagacggcccggacctacgcggaaacattaccaaccttatgcaccaccgagggacttcgctgcttatcgctccggaggtggcaagcgcggtcggaagaagctgctgtacatctttaagcaacccttcatgaggcctgtgcagactactcaagaggaggacggctgttcatgccggttcccagaggaggaggaaggcggctgcgaactgaggtctcaagttagaaaagcagctataacatcttatgagaaatctgatggagtatatacagggctcagcacgcgaaatcaggagacctatgaaactctgaagcatgagaagcccccgcagtag

SEQ ID NO: 34 - protein, 484 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVRSKRSRLLHSDYMNMTPRRPGPTRKHYQPYAPPRDFAAYRSGGGKRGRKKLLYIFKQPFMRPVQTTQEEDGCSCRFPEEEEGGCELRSQVRKAAITSYEKSDGVYTGLSTRNQETYETLKHEKPPQ

SEQ ID NO: 35 - DNA, 1515 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgcgatctaaaagaagcaggctcttgcactccgattacatgaatatgaccccgagacggcccggacctacgcggaaacattaccaaccttatgcaccaccgagggacttcgctgcttatcgctccggaggtggcaaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacagaggtctcaagttagaaaagcagctataacatcttatgagaaatctgatggagtatatacagggctcagcacgcgaaatcaggagacctatgaaactctgaagcatgagaagcccccgcagtag

SEQ ID NO: 36 - protein, 504 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVRSKRSRLLHSDYMNMTPRRPGPTRKHYQPYAPPRDFAAYRSGGGKKVAKKPTNKAPHPKQEPQEINFPDDLPGSNTAAPVQETLHGCQPVTQEDGKESRISVQERQRSQVRKAAITSYEKSDGVYTGLSTRNQETYETLKHEKPPQ

SEQ ID NO: 37 - DNA, 1509 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgaaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacagaagcgcggtcggaagaagctgctgtacatctttaagcaacccttcatgaggcctgtgcagactactcaagaggaggacggctgttcatgccggttcccagaggaggaggaaggcggctgcgaactgaggtctcaagttagaaaagcagctataacatcttatgagaaatctgatggagtatatacagggctcagcacgcgaaatcaggagacctatgaaactctgaagcatgagaagcccccgcagtag

SEQ ID NO: 38 - protein, 502 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVKKVAKKPTNKAPHPKQEPQEINFPDDLPGSNTAAPVQETLHGCQPVTQEDGKESRISVQERQKRGRKKLLYIFKQPFMRPVQTTQEEDGCSCRFPEEEEGGCELRSQVRKAAITSYEKSDGVYTGLSTRNQETYETLKHEKPPQ

SEQ ID NO: 39 - DNA, 1509 bp, Artificial Sequence
  synthetic
atgaagttgccagtacgccttctcgtcctgatgttctggattcccgcgtcctcatctgatgtcttgatgactcagacgcccctcagtctcccggtctctcttggggaccaagtgagcatcagttgtcggagtagtcagtccattgtgcattccaatgggaatacttaccttgagtggtatttgcagaagcccggacaatcaccaaagctgctcatctataaggtgtcaaaccggtttagtggggtacccgataggttctcaggtagcggctccgggactgactttacactgaagattagccgggttgaggccgaagacctcggagtctattactgctttcaaggatcacacgtcccgaggacctttgggggtggtacgaagctggagataaaacgcggttctacaagtgggagcggaaagccaggatctggcgaaggtagcaccaaaggccaggttcaactccagcaaagcggacctgaacttgtaaaaccaggagcatctgtgaagatgagctgcaaggcatctgggtacacatttaccagttaccatattcaatgggtgaaacagcgcccaggccaaggtctggaatggatagggtggatctatccgggcgatggttcaacgcaatacaacgaaaagttcaaagggaagacaacgttgactgcggataaaagtagttcaacagcatatatgctcttgtcttcacttacttctgaagatagcgccatatatttctgtgcgagggaagggacttattacgcgatggactactggggccaaggcacatccgtaaccgtaagctcaagatctgaacaaaaacttatatctgaggaagacctggggtcgaccacgactaccccggctcccaggccgccaacaccagcccccactatcgccagtcagccgctttccctgcggcccgaggcatgtaggccagcggcagggggcgccgtccatactcggggccttgatttcgcgtgtgacatctcgagcttctgggtgttggtcgttgtgggtggtgtcctggcgtgttattcactgttggttactgtggcttttataattttctgggtgaagcgcggtcggaagaagctgctgtacatctttaagcaacccttcatgaggcctgtgcagactactcaagaggaggacggctgttcatgccggttcccagaggaggaggaaggcggctgcgaactgaaaaaggtagcgaagaaacctaccaacaaggctccgcaccccaaacaggagcctcaagaaatcaactttcctgatgatttgcctgggagtaacacggcagcaccagttcaggaaacgctgcacggatgtcagccggtaacccaggaagacgggaaggaaagtaggatttcagttcaggagagacagaggtctcaagttagaaaagcagctataacatcttatgagaaatctgatggagtatatacagggctcagcacgcgaaatcaggagacctatgaaactctgaagcatgagaagcccccgcagtag

SEQ ID NO: 40 - protein, 502 aa, Artificial Sequence
  synthetic
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSRSEQKLISEEDLGSTTTTPAPRPPTPAPTIASQPLSLRPEACRPAAGGAVHTRGLDFACDISSFWVLVVVGGVLACYSLLVTVAFIIFWVKRGRKKLLYIFKQPFMRPVQTTQEEDGCSCRFPEEEEGGCELKKVAKKPTNKAPHPKQEPQEINFPDDLPGSNTAAPVQETLHGCQPVTQEDGKESRISVQERQRSQVRKAAITSYEKSDGVYTGLSTRNQETYETLKHEKPPQ

(19) United States
(12) Patent Application Publication
(10) Pub. No.: US 2022/0306723 A1
(43) Pub. Date: Sep. 29, 2022

(54) CHIMERIC ANTIGEN RECEPTOR COMPRISING CD40 CYTOPLASMIC DOMAIN AND USES THEREOF

(71) Applicant: MIGAL GALILEE RESEARCH INSTITUTE, LTD., Kiryat Shmona (IL)

(72) Inventors: Gideon GROSS, Moshav Almagor (IL); Hadas WEINSTEIN-MAROM, Upper Galilee (IL); Ofir LEVIN-PIAEDA, Moshav Kahal (IL)

(21) Appl. No.: 17/641,949

(22) PCT Filed: Sep. 10, 2020
(86) PCT No.: PCT/IL2020/050991

§ 371 Date: Mar. 10, 2022

Related U.S. Application Data
(60) Provisional application No. 62/898,704, filed on Sep. 11, 2019.

(51) Int. Cl. C07K 14/705; C12N 15/62; A61K 35/17

(52) U.S. Cl. CPC C07K 14/70596; C07K 14/70517; C07K 14/70521; C07K 14/70578; C12N 15/62; A61K 35/17; C07K 2319/03

(57) ABSTRACT

A nucleic acid molecule encoding an activating chimeric antigen receptor (aCAR) comprising at least one signal transduction element derived from CD40 is provided.

### CROSS REFERENCE TO RELATED APPLICATIONS

The present application claims the benefit of priority to U.S. Provisional Patent Application No. 62/898,704 filed Sep. 11, 2019, the contents of which are incorporated by reference herein in their entirety.

### FIELD OF THE INVENTION

The present invention relates in general to activating chimeric antigen receptors (aCARs) and their use in immunotherapy of cancer.

### BACKGROUND OF THE INVENTION

In the late 1980s and early 1990s, double- and single-chain CARs were introduced as new genetic means to redirect T cells at will, paving the way to the entire field of CAR-T cell therapy of cancer [1,2]. In 2017 the first CAR-T products were approved by the FDA for the treatment of B cell acute lymphoblastic leukemia and non-Hodgkin lymphoma and approximately four hundred clinical trials are currently examining CAR therapy in a wide range of hematologic malignancies and solid tumors (see our review [3], [4,5] and https://clinicaltrials.gov/).

The low response rates observed in solid tumors underscore the need in enhancing the tumoricidal activity of CAR-T cells for improving their therapeutic efficacy. Aside from the apparent lack of suitable tumor antigens, CAR-T cells face major obstacles posed by both extrinsic and intrinsic factors. Extrinsically, the transferred T cells are confronted by the resilient tumor microenvironment, which often recruits immune suppressor cells, including regulatory T cells (Tregs), myeloid derived suppressor cells and tumor-associated macrophages and exploits diverse evasion mechanisms to prevent T cell access and avoid an immunological attack [6]. Prominent among the intrinsic hurdles are the limited persistence and proliferative capacity of the transfused T cells, their functional exhaustion following lengthy ex-vivo propagation, the acquisition of an unfavorable terminal effector T cell differentiation state, antigen-induced cell death (AICD) as a result of prolonged exposure to antigen and the uncontrolled production of tonic signaling, which may impede T cell reactivity and survival [7,8].

In attempt to overcome these limitations, a huge amount of effort is put in recent years into optimizing the signaling moieties incorporated into the intracellular portion of 2^{nd} and 3^{rd} generation CARs, which largely govern the clinical outcome of CAR-T cell therapy. A variety of costimulatory signaling elements and their combinations, capable of augmenting diverse aspects of T cell function and lifespan, have been explored along this route, including CD28, 4-1BB, OX40, ICOS, CD27, CD244, CD80 and 4-1BBL [8-10]. The most widely explored of these are undoubtedly CD28 and 4-1BB.

Engagement of the key costimulatory receptor CD28 with B7 proteins on antigen-presenting cells (APCs) is mandatory for priming of naïve T cells, signaling enhanced TCR-induced proliferation, differentiation and acquisition of diverse effector functions [11-13]. 4-1BB (CD137), a member of the tumor necrosis factor receptor (TNFR) family is expressed on activated human T cells. The TNFR family also includes OX40 and CD27, whose signaling elements have similarly been assessed in advanced CAR designs. The cytosolic portions of these TNFRs bear structural similarities and they all signal through adaptor TNFR-associated factor (TRAF) proteins via the NFκB, p38 MAPK or JNK/SAPK pathways [14].

4-1BB ligation by cognate ligand or soluble agonists promotes T cell survival by counteracting apoptosis, induces cell division, augments Th1 cytokine production, protects T cells from AICD, drives memory formation and confers resistance to Treg suppression [11,15]. Not surprisingly, the signaling domains of CD28 and 4-1BB have been extensively explored as the costimulatory components of 2^{nd} and 3^{rd} generation CARs.

Numerous studies of 2^{nd} generation CARs have revealed marked differences in the effects exerted by the CD28 and 4-1BB signaling domains, reflecting their distinct physiological roles. [8,11]. For example, CD28 was shown to support the rapid acquisition of effector functions and tumor eradication capacity but only limited persistence, both in apparent contrast to 4-1BB (e.g., [10,16,17]). While CD28 mostly induces CAR-T cell differentiation into the effector-memory type, 4-1BB preferentially drives central memory formation [18]. Surprisingly, engrafting these two costimulatory domains in tandem to create 3^{rd} generation CARs does not necessarily result in improved therapeutic activity in-vivo (see [8] for a recent review). Moreover, it is becoming increasingly clear that not all physiological functions of native CD28 and 4-1BB are preserved in CARs [11]. Critically, contradicting reports on functional outcomes may merely reflect different experimental contexts. For example, 4-1BB-mediated tonic signaling was reported to exert either positive [9] or negative [19] effects on CAR-T cell survival and functionality.

Thus, there still remains an unmet need for improved aCARS for use in immunotherapy of cancer.

### SUMMARY OF THE INVENTION

In one aspect, the present invention provides a nucleic acid molecule comprising a nucleotide sequence encoding an activating chimeric antigen receptor (aCAR) comprising: (i) an extracellular binding domain capable of binding to an antigen; (ii) a transmembrane domain; (iii) an intracellular domain; and (iv) a flexible hinge or stalk domain linking the extracellular binding domain and the transmembrane domain, said flexible hinge or stalk domain comprising a cysteine residue capable of forming a cysteine bridge thereby forming an aCAR homodimer, wherein said intracellular domain is selected from:

(a) an intracellular domain comprising at least one signal transduction element derived from CD40 and a second amino acid sequence comprising at least one signal transduction element derived from an FcR gamma (γ) chain, CD3 zeta (ζ) chain, or CD3 eta (η) chain, and lacking a MyD88 polypeptide, 2A self-cleaving peptide or a dimerizing domain;

(b) an intracellular domain comprising at least one signal transduction element derived from CD40 and a third amino acid sequence comprising at least one signal transduction element derived from CD28 or 4-1BB; and

(c) an intracellular domain comprising a first amino acid sequence comprising at least one signal transduction element derived from CD40, a second amino acid sequence comprising at least one signal transduction element derived from an FcR gamma (γ) chain, CD3 zeta (ζ) chain, or CD3 eta (η) chain and a third amino acid sequence comprising at least one signal transduction element derived from CD28; and

(d) an intracellular domain comprising a first amino acid sequence comprising at least one signal transduction element derived from CD40 and lacking a MyD88 polypeptide or a truncated MyD88 polypeptide lacking the Toll/IL-1 receptor domain (TIR) domain and may alternatively or further lack a myristoylation-targeting sequence or a dimerizing domain, such as an FKBP12v36 domain.

In another aspect, the present invention provides a composition comprising the nucleic acid molecule of any one of any one of the above embodiments.

In an additional aspect, the present invention provides a vector comprising the nucleic acid molecule of any one of the above embodiments.

In yet another aspect, the present invention provides a mammalian T cell comprising the nucleic acid molecule of any one of the above embodiments, or the DNA vector of any one of the above embodiments.

In yet an additional aspect, the present invention provides a method of preparing allogeneic or autologous aCAR T cells, the method comprising contacting T cells with the nucleic acid molecule of any one of any one of the above embodiments; or a DNA vector as defined above, thereby preparing allogeneic or autologous aCAR T cells.

In a further aspect, the present invention provides a method of treating or preventing a disease, disorder or condition in a subject, comprising administering to said subject the mammalian T cell of any one of the above embodiments, wherein said T cell is a CD8⁺ effector T cell and said disease, disorder or condition is selected from a solid tumor, hematologic malignancy, melanoma, infection with a virus; or said T cell is a CD4⁺ regulatory T cell (Treg) and said disease, disorder or condition is manifested in excessive activity of the immune system, such as an autoimmune disease, allergy, asthma, and organ and bone marrow transplantation.

### BRIEF DESCRIPTION OF THE DRAWINGS

The patent or application file contains at least one drawing executed in color or grayscale. Copies of this patent or patent application publication with color or grayscale drawings will be provided by the Office upon request and payment of necessary fee.

FIG. 1 shows a schematic representation of the eight CARs, where “Li” refers to the first flexible peptide linker of the single chain variable fragment, and “T” refers to the Myc tag.

FIG. 2 shows CAR surface expression. K562 cells were transfected by electroporation with 10 μg of each of the indicated mRNAs. Twenty-four hours later cells were subjected to flow cytometry analysis for expression of the Myc tag. Black histograms, irrelevant mRNA; grey histograms, CARs. See FIG. 1 for key to CAR names.

FIGS. 3A-B shows CAR functionality. (A) Antigen-specific activity of the new CARs (see FIG. 1 for key to CAR names). B3Z T cells possessing the reporter NFAT-LacZ gene were electroporated with each of the indicated mRNAs. Seven hours post-electroporation cells were incubated at 1:1 ratio with 579 melanoma cells (checkered), 579-A2 melanoma cells (black) or no cells (white), and 24 hours later cell lysates were subjected to the colorimetric CPRG assay. (B) Activation of the NF-κB signaling pathway. HEK293T cells were transfected with the NF-κB-luciferase reporter plasmid and 24 hours later with mRNA encoding each of the indicated constructs. Histograms show relative luminescence units (RLU) in cell lysates. 2C11, activation by anti-CD3 mAb; Irr., irrelevant mRNA; P.C., positive control, constitutively active CD40 (caCD40, (7)). Results are representative of three independent experiments.

FIGS. 4A-F show antigen-specific activation of human CD8 T cells. CD8 T cells of Donor I (A-C) and Donor II (D-F) that were electroporated with CAR mRNA and subjected to expression and function analyses (see FIG. 1 for key to CAR names). ELISA for the secretion of pro-inflammatory cytokines IFN-γ (A, D), TNF-α (B, E), GM-CSF (C, F).

FIGS. 5A-B show LDH assay for target cell (579 melanoma cells (white) or 579-A2 melanoma cells (black)) killing by cells obtained from Donor I (A) and Donor II (B). Results shown in ‘A’ represent three, and in ‘B’ two, independent experiments, all performed separately for CD8 T cells of each of the two donors.

### DETAILED DESCRIPTION OF THE INVENTION

CD40 is a member of the TNFR family and is mainly expressed by professional APCs. A number of studies suggest that CD40 is functionally expressed also by T cells. The direct T cell stimulatory capacity of CD40 was manifested in a wide range of effects including differentiation, memory formation, improvement of functional avidity, upregulation of anti-apoptotic signals and decreasing pro-apoptotic ones, rescue from exhaustion and acquisition of resistance to Treg-mediated suppression [20-22]. Yet, other studies failed to confirm these observations and the immunological role played by T cell-expressed CD40 under physiological conditions is still elusive. Nevertheless, recently, the potent costimulatory capacity of the CD40 signaling domain has successfully been recruited to native unmodified T cells [23-25]. In contrast, others have found that CD40 signaling domains are inactive in the context of aCARs unless they are conjugated with self-assembly and myD88 domains [26-28].

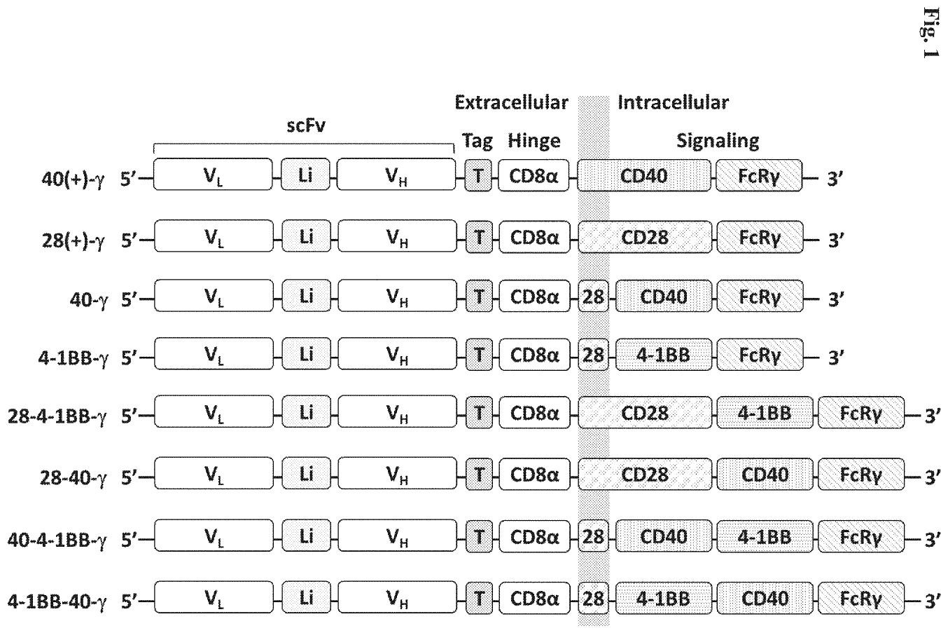

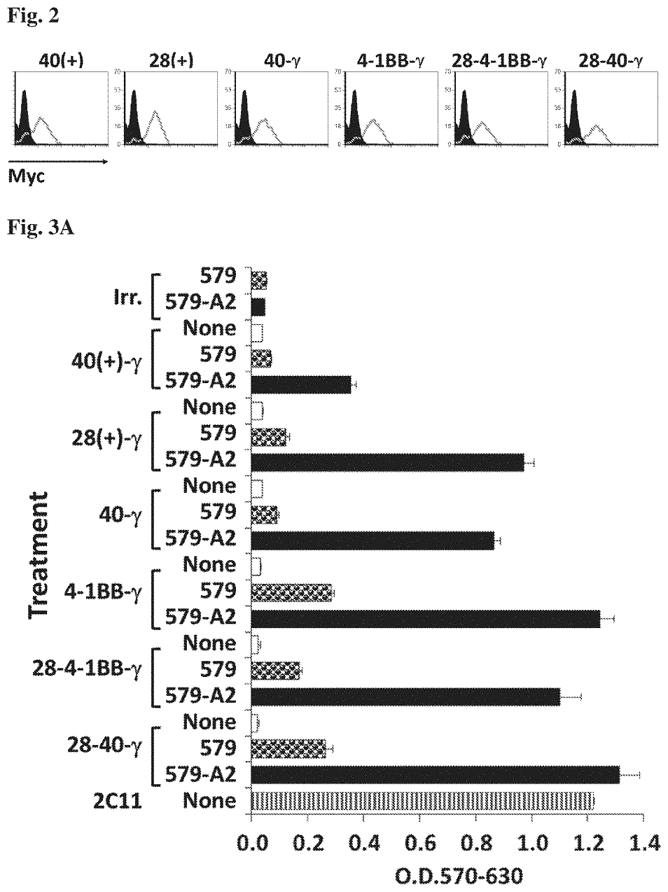

The inventors of the present invention have constructed a series of new anti-HLA-A2 CARs harboring either the intracellular signaling domain of CD40 or of 4-1BB, with or without the intracellular signaling domain of CD28, and examined these CARs in mRNA-electroporated human CD8 T cells. Schematic presentation of the series of constructs examined is depicted in FIG. 1 and detailed in Table 1. It was unexpectedly found that the mere incorporation of the CD40 signaling domain in the intracellular portion of the different CARs led to spontaneous activation of the NF-κB signaling pathway, which was consistently higher than that induced by the corresponding CARs harboring the 4-1BB signaling domain (FIG. 2B). Furthermore, although not even traces of CD40 expression could be detected in any of the human T cells previously tested by us, the CD40 signaling domain was fully potent in these T cells, as manifested by the introduction of either caCD40 or of native CD40, followed by CD4OL stimulation [24].

In one aspect, the present invention a nucleic acid molecule comprising a nucleotide sequence encoding an activating chimeric antigen receptor (aCAR) comprising: (i) an extracellular binding domain capable of binding to an antigen; (ii) a transmembrane domain; (iii) an intracellular domain; and (iv) a flexible hinge or stalk domain linking the extracellular binding domain and the transmembrane domain, said flexible hinge or stalk domain comprising a cysteine residue capable of forming a cysteine bridge thereby forming an aCAR homodimer, wherein said intracellular domain is selected from:

(a) an intracellular domain comprising at least one signal transduction element derived from CD40 and a second amino acid sequence comprising at least one signal transduction element derived from an FcR gamma (γ) chain, CD3 zeta (ζ) chain, or CD3 eta (η) chain, and lacking a MyD88 polypeptide, 2A self-cleaving peptide or a dimerizing domain;

(b) an intracellular domain comprising at least one signal transduction element derived from CD40 and a third amino acid sequence comprising at least one signal transduction element derived from CD28 or 4-1BB; and

(c) an intracellular domain comprising a first amino acid sequence comprising at least one signal transduction element derived from CD40, a second amino acid sequence comprising at least one signal transduction element derived from an FcR gamma (γ) chain, CD3 zeta (ζ) chain, or CD3 eta (η) chain and a third amino acid sequence comprising at least one signal transduction element derived from CD28; and

(d) an intracellular domain comprising a first amino acid sequence comprising at least one signal transduction element derived from CD40 and lacking a MyD88 polypeptide or a truncated MyD88 polypeptide lacking the Toll/IL-1 receptor domain (TIR) domain and may alternatively or further lack a myristoylation-targeting sequence or a dimerizing domain, such as an FKBP12v36 domain [29].

The signal transduction element derived from CD40 may be a tumor necrosis factor receptor (TNFR)-associated factor (TRAF)-binding domain, e.g. TRAF2, TRAF3, TRAFS and TRAF6 binding domain. TRAF2, 3 and 5 usually have overlapping binding motifs, whereas TRAF6 has a distinct interacting motif on these receptors, and TRAF1 binds to the CD40 signal transduction element via TRAF2 [30].

In certain embodiments, the signal transduction element derived from the FcR gamma (γ) chain, CD3 zeta (ζ) chain, or CD3 eta (η) chain is an immunoreceptor tyrosine-based activation motif (ITAM). This motif contains a tyrosine separated from a leucine or isoleucine by any two other amino acids, giving the signature YxxL/I. Two of these signatures are typically separated by between 6 and 8 amino acids in the cytoplasmic tail of the molecule (YxxL/Ix(6-8)YxxL/I).

A T cell or cell population comprising a T cell expressing the aCAR of the present invention has an increased cytotoxic activity against a cell having the target antigen on the surface, as compared to a T cell or cell population comprising a T cell expressing an aCAR whose intracellular domain consists of an intracellular domain of CD28 and CD3, 4-1BB and CD3, or CD28 and 4-1BB.

In certain embodiments, the extracellular binding domain comprises (i) an antibody, derivative or fragment thereof, such as a humanized antibody; a human antibody; a functional fragment of an antibody; a single-domain antibody, such as a Nanobody; a recombinant antibody; and a single chain variable fragment (ScFv); (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer.

In particular embodiments, the extracellular binding domain comprises an ScFv. In certain embodiments, the ScFv comprises a Variable Light chain (VL) and a Variable Heavy chain (VH) linked by a first flexible peptide linker, e.g. of SEQ ID NO: 12.

In a certain embodiment, the transmembrane domain of the aCAR is selected from the transmembrane domain of CD28, CD40, CD3-η, TLR1, TLR2, TLR4, TLR5, TLR9, and Fc receptor.

In certain embodiments, the transmembrane domain is the CD28 transmembrane domain, e.g. of SEQ ID NO: 16.

In certain embodiments, the transmembrane domain is the CD40 transmembrane domain, e.g. of SEQ ID NO: 22.

In certain embodiments, the first amino acid sequence is the complete intracellular domain of CD40, e.g. of SEQ ID NO: 17.

In certain embodiments, the at least one signal transduction element of the second amino acid sequence is derived from an FcRγ chain.

In certain embodiments, the second amino acid sequence is the complete intracellular domain of an FcRγ chain, e.g. of SEQ ID NO: 18.

In certain embodiments, the third amino acid sequence is the complete intracellular domain of CD28, e.g. of SEQ ID NO: 20.

In certain embodiments, the flexible hinge or stalk comprises a polypeptide selected from a hinge region of CD8α or CD8β. The sequence and structure of these hinge domains are well-characterized (e.g. Wong et al. [31]). The flexible hinge or stalk may further by selected from a hinge region of a heavy chain of IgG, and a hinge region of a heavy chain of IgD.

In certain embodiments, the flexible hinge domain is the hinge domain of CD8α, e.g. a complete flexible hinge domain, optionally altered by the addition of two Ser residues at its C-terminus (to form an XhoI restriction site), such as in the sequence of SEQ ID NO: 15.

In particular embodiments, the nucleic acid molecule of any one of the above embodiments comprises a nucleotide sequence encoding an aCAR comprising an extracellular binding domain comprising (i) an antibody, derivative or fragment thereof, such as a humanized antibody; a human antibody; a functional fragment of an antibody; a single-domain antibody, such as a Nanobody; a recombinant antibody; and a single chain variable fragment (ScFv); (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer; said transmembrane domain is selected from the transmembrane domain of CD28, CD40, CD3-ζ TLR1, TLR2, TLR4, TLR5, TLR9, and Fc receptor; the first amino acid sequence is the complete intracellular domain of CD40, e.g. of SEQ ID NO: 17; the at least one signal transduction element of the second amino acid sequence is derived from an FcRγ chain; the third amino acid sequence, when present, is the complete intracellular domain of CD28, e.g. of SEQ ID NO: 20; and the flexible hinge comprises a polypeptide selected from a hinge region of CD8α, CD8β, a hinge region of a heavy chain of IgG, and a hinge region of a heavy chain of IgD.

In certain embodiments, the aCAR of the previous embodiment comprises an extracellular binding domain comprising an ScFv; the transmembrane domain is the transmembrane domain of CD28 e.g. of SEQ ID NO: 16; the second amino acid sequence is the complete intracellular domain of an FcRγ chain, e.g. of SEQ ID NO: 18; and the flexible hinge domain is the flexible hinge domain of CD8α.

In certain embodiments, the intracellular domain of any one of the above aCARs comprises or essentially consists of a tandem arrangement of the complete intracellular domains of CD40-FcRγ.

In certain embodiments, the intracellular domain of any one of the above aCARs comprises or essentially consists of a tandem arrangement of the complete intracellular domains of CD28-CD40-FcRγ, wherein the intracellular domain of CD28 is optionally linked to the intracellular domain of CD40 via a short oligopeptide linker.

Or in other words, the tandem arrangement is selected from a polypeptide comprising the complete intracellular domains of [N-terminus-CD28]-[optional short oligopeptide linker]-[CD40-FcRγ-C-terminus]; and [N-terminus- CD40]-[FcRγ-C-terminus]. The order of appearance of the different domains from N- to C-terminus can be different, e.g. [N-terminus-CD40]-[optional short oligopeptide linker]-[CD28-FcRγ-C-terminus].

In certain embodiments, the intracellular domain of any one of the above aCARs comprises or essentially consists of the complete intracellular domains of 4-1BB, CD40, and FcRγ. For example, the aCAR may comprise an ScFv; the transmembrane domain of CD28; the complete intracellular domain of CD40, the complete intracellular domain of 4-1BB, the complete intracellular domain of an FcRγ chain; and the flexible hinge domain of CD8α.

Flexible peptide linkers are well-known in the art. Empirical linkers designed by researchers are generally classified into three categories according to their structures: flexible linkers, rigid linkers, and in vivo cleavable linkers as defined e.g. in [32-34], each one of which is incorporated by reference as if fully disclosed herein.

The structure of the flexible short oligopeptide linker is selected from any one of the linkers disclosed in [32-34]. In principle, to provide flexibility, the linkers are generally composed of small, non-polar (e.g. Gly) or polar (e.g. Ser or Thr) amino acids, such an underlying sequence of alternating Gly and Ser residues. Solubility of the linker and associated signal transduction elements may be enhanced by including charged residues; e.g. two positively charged residues (Lys) and one negatively charged residue (Glu). The linker may vary from 2 to 31 amino acids, optimized for each condition so that the linker does not impose any constraints on the conformation or interactions of the linked partners in lengths.

In a certain embodiment, the flexible short oligopeptide linker has the amino acid sequence Gly-Gly-Gly.

In certain embodiments, the intracellular domain comprises or essentially consists of a tandem arrangement of the complete intracellular domains of [CD28]-[short oligopeptide linker]-[CD40]-[FcRγ] (from N- to C-terminus). (28-40-γ)

In certain embodiments, the aCAR comprises a tandem arrangement of (from N- to C-terminus) [ScFv]-[hinge region of CD8α-CD28 transmembrane domain]-[intracellular domain essentially consisting of a tandem arrangement of the complete intracellular domains of CD40-FcRγ]. (40-γ)

In certain embodiments, the aCAR comprises a tandem arrangement of (from N- to C-terminus) [ScFv]-[hinge region of CD8α-CD28 transmembrane domain]-[intracellular domain essentially consisting of a tandem arrangement of the complete intracellular domains of CD28-CD40-FcRγ], wherein the intracellular domain of CD28 is optionally linked to the intracellular domain of CD40 via a linker.

In certain embodiments, the DNA or amino-acid sequence of each one of the different domains of the aCAR is the human sequence.

In certain embodiments, the aCAR, excluding the extracellular binding domain, comprises the combined amino acid sequences of SEQ ID NOs: 15+16+20+Gly-Gly-Gly+17+18; or 15+16+17+18.

Non-limiting examples of complete sequences of DNA encoding aCARs of the present invention are set forth in SEQ ID NOs: 25, 27, 29, 31, 33, 35, 37, and 39; and non-limiting examples of complete amino acid sequences of aCARs of the present invention are set forth in 26, 28, 30, 32, 34, 36, 38, and 40. It should be made clear that these are examples disclosed for the sole purpose of teaching one specific way of making the present invention, which can be easily adapted by a person skilled in the art to fulfill different experimental demands, e.g. by the introduction or abolishment of enzyme restriction sites or short flexible oligopeptide linkers as required, or by synonymous changes in the DNA sequence to improve expression.

The different domains, and full sequence, of the aCARs defined above may have amino acid sequences that have at least 40%, at least 50%, at least 60%, at least 70%, at least 80%, at least 85%, at least 90%, or at least 95, 96, 97, 98, or 99% identity to the sequences defined in SEQ ID NO: 15, 16, 17, 18, 20, 26, 28, 30, 32, 34, 36, 38, and 40 and other combined sequences recited above, respectively, as long as the aCAR is active, i.e. is capable of activating a T cell in an antigen-dependent manner.

The nucleotide sequences encoding the various domains of the aCAR defined above comprise all redundant nucleotide sequences encoding the amino acid sequences of these domains as well as similar sequences encoding for active aCARs. Thus, the nucleotide sequences encoding for amino acid sequences of SEQ ID NO: 15, 16, 17, 18, 20, 26, 28, 30, 32, 34, 36, 38, and 40 are as set forth in SEQ ID NO: 6, 7, 8, 9, 20, 25, 27, 29, 31, 33, 35, 37, and 39, respectively; or any other redundant sequence encoding for identical amino acid sequences. Furthermore, the nucleotide sequences may have at least 40%, at least 50%, at least 60%, at least 70%, at least 80%, at least 85%, at least 90%, or at least 95, 96, 97, 98, or 99% identity to the sequences defined in SEQ ID NO: 6, 7, 8, 9, 19, 25, 27, 29, 31, 33, 35, 37, and 39, and the nucleotide sequences encoding for other combined amino acid sequences recited above, respectively, as long as the encoded aCAR is active, i.e. is capable of activating a T cell in an antigen-dependent manner.

In certain embodiments, the intracellular domain of (c) or (d) of any one of the above embodiments lacks MyD88 polypeptide or a truncated MyD88 polypeptide lacking the Toll/IL-1 receptor domain (TIR) domain and may alternatively or further lack a myristoylation-targeting sequence or a dimerizing domain, such as an FKBP12v36 domain.

In certain embodiments, the intracellular domain of (a) (b), (c) or (d) of any one of the above embodiments lacks a self-cleaving peptide, such as 2A self-cleaving peptide including any one of P2A, E2A, F2A and T2A.

In another aspect, the present invention provides a composition comprising the nucleic acid molecule of any one of any one of the above embodiments.

Matuskova and Durinikova [35] teach that there are two systems for the delivery of transgenes into a cell—viral and non-viral. The non-viral approaches are represented by polymer nanoparticles, lipids, calcium phosphate, electroporation/nucleofection or biolistic delivery of DNA-coated microparticles or mRNA. The non-viral approach also provides transposon systems, such as the transposon system commonly known as “Sleeping Beauty” (for protocols using Sleeping Beauty transposons see for example [36].

The viral approach provides two main types of vectors that can be used in accordance with the present invention depending on whether the DNA is integrated into chromatin of the host cell or not. Retroviral vectors such as those derived from gammaretroviruses or lentiviruses persist in the nucleus as integrated provirus and reproduce with cell division. Other types of vectors (e.g. those derived from herpesviruses or adenoviruses) remain in the cell in the episomal form.

In an additional aspect, the present invention provides a vector comprising the nucleic acid molecule of any one of the above embodiments.

In certain embodiments, the vector of any one of the above embodiments is a DNA vector, such as a plasmid or viral vector; or a non-viral vector, such as a polymer nanoparticle, lipid, calcium phosphate, DNA-coated microparticle or transposon.

In certain embodiments, the DNA vector is a viral vector selected from a modified virus derived from a virus selected from the group consisting of a retrovirus, lentivirus, gammavirus, adenovirus, adeno-associated virus, poxvirus, alphavirus, and herpes virus.

In yet another aspect, the present invention provides a mammalian T cell comprising the nucleic acid molecule of any one of the above embodiments, or the DNA vector of any one of the above embodiments.

In certain embodiments, the mammalian T cell defined above is a CD4⁺ helper T cell or regulatory T cell (Treg); or it may be a CD8⁺ effector T cell.

In certain embodiments, the mammalian T cell defined above is expressing on its surface said aCAR.

In certain embodiments, the mammalian T cell defined above is a human T cell. In yet an additional aspect, the present invention provides a method of preparing allogeneic or autologous aCAR T cells, the method comprising contacting T cells with the nucleic acid molecule of any one of any one of the above embodiments; or a vector as defined above, thereby preparing allogeneic or autologous aCAR T cells.

The immune cells may be transfected with the appropriate nucleic acid molecule described herein by e.g. RNA transfection or by incorporation in a plasmid fit for replication and/or transcription in a eukaryotic cell or a viral vector. In certain embodiments, the vector is a viral vector selected from a modified virus derived from a virus selected from the group consisting of a retrovirus, lentivirus, gammavirus, adenovirus, adeno-associated virus, pox virus, alphavirus, and herpes virus.

Combinations of retroviral vector and an appropriate packaging line can also be used, where the capsid proteins will be functional for infecting human cells. Several amphotropic virus-producing cell lines are known, including PA12 [37], PA317 [38] and CRIP [39]. Alternatively, non-amphotropic particles can be used, such as, particles pseudotyped with VSVG, RD 114 or GAL V envelope. Cells can further be transduced by direct co-culture with producer cells, e.g., by the method of Bregni, et ai. [40], or culturing with viral supernatant alone or concentrated vector stocks, e.g., by the method of Xu, et al. [41]; and Hughes, et al. [42]. In a further aspect, the present invention provides a method of studying T cell signal transduction pathways and the effect of intracellular signaling domains on activation and ligand-dependent cell killing abilities; for example, by assessing the relative physical positioning of the different signaling and activation domains along the intracellular portion.

In a further aspect, the present invention provides a method of treating or preventing a disease, disorder or condition in a subject, comprising administering to said subject the mammalian T cell of any one of the above embodiments, wherein said T cell is a CD8⁺ effector T cell and said disease, disorder or condition is selected from a solid tumor, hematologic malignancy, melanoma, infection with a virus; or said T cell is a CD4⁺ regulatory T cell (Treg) and said disease, disorder or condition is manifested in excessive activity of the immune system, such as an autoimmune disease, allergy, asthma, and organ and bone marrow transplantation.

For example, the autoimmune disease may be selected from type 1 diabetes; rheumatoid arthritis; psoriasis; psoriatic arthritis; multiple sclerosis; systemic lupus erythematosus; inflammatory bowel disease, such as Crohn's disease and ulcerative colitis; Addison's disease; Graves' disease; Sjogren's syndrome; Hashimoto's thyroiditis; myasthenia gravis; vasculitis; pernicious anemia; celiac disease; and atherosclerosis.

In certain embodiments, the subject is preferably human and said mammalian Treg is allogeneic or autologous human T cell.

Definitions

The term “allogeneic” as used herein refers to tissues, organs or cells that are genetically dissimilar from, and hence immunologically incompatible with, a host receiving them, although from individuals of the same species.

The term “autologous” as used herein refers to tissues, organs or cells obtained from the same individual receiving them.

As used herein, the terms “subject” or “individual” or “animal” or “patient” or “mammal,” refers to any subject, particularly a mammalian subject, for whom diagnosis, prognosis, or therapy is desired, for example, a human.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. In certain embodiments, the pharmaceutical composition is adapted for oral administration.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

The term “therapeutically effective amount” as used herein means an amount of the nucleic acid sequence/molecule or vector that will elicit the biological or medical response of a tissue, system, animal or human that is being sought, i.e. treatment of a disease associated with or caused by a cell state, such as cancer. The amount must be effective to achieve the desired therapeutic effect as described above, depending inter alia on the type and severity of the condition to be treated and the treatment regime. The therapeutically effective amount is typically determined in appropriately designed clinical trials (dose range studies) and the person skilled in the art will know how to properly conduct such trials to determine the effective amount. As generally known, an effective amount depends on a variety of factors including the affinity of the ligand to the receptor, its distribution profile within the body, a variety of pharmacological parameters such as half-life in the body, on undesired side effects, if any, and on factors such as age and gender, etc.

The transition phrase “consisting essentially of” or “essentially consisting of”, when referring to an amino acid or nucleic acid sequence, refers to the a sequence that includes the listed sequence and is open to present or absent unlisted sequences that do not materially affect the basic and novel properties of the protein itself or the protein encoded by the nucleic acid sequence.

Unless otherwise indicated, all numbers used in this specification are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

### Equivalents and Scope

Those skilled in the art will recognize, or be able to ascertain, using no more than routine experimentation, many equivalents to the certain embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes some embodiments in which exactly one member of the group is present in, employed in, or otherwise relevant to a given product or process. The disclosure includes some embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The invention will now be illustrated by the following non-limiting Examples.

### EXAMPLES

Materials and Methods

Cell Lines

B3Z is an OVA₂₅₇₋₂₆₄-specific, H-2K^{b}-restricted CTL hybridoma harboring the nuclear factor of activated T-cells (NFAT)-lacZ inducible reporter gene. HEK-293T is a human embryonic kidney cell line expressing T-antigen.

M579 (579) is an HLA-A2-negative melanoma cell line and 579-A2 is a 579 transfectant expressing HLA-A2. B3Z is an OVA257-264-H-2Kb-specific mouse T cell hybridoma harboring the nuclear factor of activated T-cells (NFAT)-lacZ inducible reporter gene.

Human T Cell Culture

Human peripheral blood mononuclear cells (PBMCs) were obtained from the MDA National Blood Services (Tel-Hashomer, Israel).

Human lymphocytes were cultured in complete RPMI 1640 medium supplemented with 10% heat-inactivated human AB serum (Sigma-Aldrich, Saint Louis, Mo.) or FCS, 300 and 6,000 IU/ml recombinant human IL-2 for PBMCs and Tumor Infiltrating Leukocyte (TIL) cultures, respectively (rhlL-2; Chiron, Amsterdam, The Netherlands), 2 mmol/L L-glutamine, 1 mmol/L sodium pyruvate, 1% nonessential amino acids, 25 mM HEPES, 50 μM 2-mercaptoethanol and combined antibiotics.

CD8 T cells were separated by positive selection using magnetic beads (BD), grown for 3-4 days in the presence of soluble OKT3 and anti-CD28 mAbs and 1000 U/ml recombinant human IL-2 (rhlL-2, Chiron).

Plasmids and Gene Cloning

CAR genes were assembled via modular restriction cloning as DNA templates for in-vitro transcription in the pGEM4Z/EGFP/A64 vector [43]. For the NF-κB assay we used an NF-κB-Luciferase reporter plasmid.

The constructs include the sequences as detailed in Table 1:

TABLE 1
Sequences used in constructs/fusion proteins

| | TYPE | | | | | | | | | |
|---|---|---|---|---|---|---|---|---|---|---|
| SEQ. | (DNA/ | | | | | | 28-4- | 28- | 40-4- | 4-1BB- |
| ID. N0. | PROTEIN) | DOMAIN | 40(+)-γ | 28(+)-γ | 40-γ | 4-1BB-γ | 1BB-γ | 40-γ | 1BB-γ | 40-γ |
| 1. | DNA | 5′ nontranslated | √ | √ | √ | √ | √ | √ | √ | √ |
| 2. | DNA | Leader peptide + | √ | √ | √ | √ | √ | √ | √ | √ |
| | | Variable Heavy chain | | | | | | | | |
| 3. | DNA | Linker | √ | √ | √ | √ | √ | √ | √ | √ |
| 4. | DNA | Variable Light chain | √ | √ | √ | √ | √ | √ | √ | √ |
| 5. | DNA | Myc Tag | √ | √ | √ | √ | √ | √ | √ | √ |
| 6. | DNA | CD8α hinge | √ | √ | √ | √ | √ | √ | √ | √ |
| 7. | DNA | CD28 transmembrane | | √ | √ | √ | √ | √ | √ | √ |
| 8. | DNA | CD40 intracellular | √ | | √ | | √ | √ | √ | √ |
| 9. | DNA | FcRγ intracellular | √ | √ | √ | √ | √ | √ | √ | √ |
| 10. | DNA | 3′ nontranslated | √ | √ | √ | √ | √ | √ | √ | √ |
| 11. | Protein | Leader peptide + | √ | √ | √ | √ | √ | √ | √ | √ |
| | | Variable Heavy chain | | | | | | | | |
| 12. | Protein | Linker | √ | √ | √ | √ | √ | √ | √ | √ |
| 13. | Protein | Variable Light chain | √ | √ | √ | √ | √ | √ | √ | √ |
| 14. | Protein | Myc Tag | √ | √ | √ | √ | √ | √ | √ | √ |
| 15. | Protein | CD8α hinge | √ | √ | √ | √ | √ | √ | √ | √ |
| 16. | Protein | CD28 transmembrane | | √ | √ | √ | √ | √ | √ | √ |
| 17. | Protein | CD40 intracellular | √ | | √ | | √ | √ | √ | √ |
| 18. | Protein | FcRγ intracellular | √ | √ | √ | √ | √ | √ | √ | √ |
| 19. | DNA | CD28 intracellular | | √ | | | √ | √ | | |
| 20. | Protein | CD28 intracellular | | √ | | | √ | √ | | |
| 21. | DNA | CD40- | √ | | | | | | | |
| | | transmembrane | | | | | | | | |
| 22. | Protein | CD40- | √ | | | | | | | |
| | | transmembrane | | | | | | | | |
| 23. | DNA | 4-1BB intracellular | | | | √ | √ | | √ | √ |
| 24. | Protein | 4-1BB intracellular | | | | √ | √ | | √ | √ |
| 25. | DNA | complete 40(+)-γ | √ | | | | | | | |
| 26. | Protein | complete 40(+)-γ | √ | | | | | | | |
| 27. | DNA | complete 28(+)-γ | | √ | | | | | | |
| 28. | Protein | complete 28(+)-γ | | √ | | | | | | |
| 29. | DNA | complete 40-γ | | | √ | | | | | |
| 30. | Protein | complete 40-γ | | | √ | | | | | |
| 31. | DNA | complete 4-1BB-γ | | | | √ | | | | |
| 32. | Protein | complete 4-1BB-γ | | | | √ | | | | |
| 33. | DNA | complete 28-4-1BB-γ | | | | | √ | | | |
| 34. | Protein | complete 28-4-1BB-γ | | | | | √ | | | |
| 35. | DNA | complete 28-40-γ | | | | | | √ | | |
| 36. | Protein | complete 28-40-γ | | | | | | √ | | |
| 37. | DNA | complete 40-4-1BB-γ | | | | | | | √ | |
| 38. | Protein | complete 40-4-1BB-γ | | | | | | | √ | |
| 39. | DNA | complete 4-1BB-40-γ | | | | | | | | √ |
| 40. | Protein | complete 4-1BB-40-γ | | | | | | | | √ |

In-Vitro Transcription of mRNA

Template plasmids were linearized with Spel. Transcription and capping reactions were carried out using AmpliCap-Max T7 High Yield Message Maker Kit (Epicentre Biotechnologies, Madison, Wis.). The mRNA product was purified by DNase-I digestion, followed by LiCl precipitation, according to the manufacturer's instructions. The quality of the mRNA product was assessed by agarose gel electrophoresis and concentration was determined by spectrophotometric analysis. Purified mRNA was stored at −80° C. in small aliquots.

mRNA Electroporation of Human T Cells

Electroporation was performed with ECM830 Electro Square Wave Porator (Harvard Apparatus BTX, Holliston, Mass.) at LV mode, single pulse, 500 V, 1 msec, or Gene Pulser Xcell (Bio-Rad Laboratories, Hercules, Calif.) using a square-wave pulse, 500 V, 1 msec in cold 2 mm cuvettes as follows: Stimulated CD8 T cells and TILs were washed twice with OptiMEM medium (Gibco, Grand Island, N.Y.) and resuspended in OptiMEM at a final concentration of 3×10⁷/ml. For electroporation 0.1 to 0.4 ml pre-chilled cells (5 minutes on ice) were mixed with the required amount of in-vitro-transcribed mRNA. In transfection experiments involving more than one mRNA species, the appropriate amount of irrelevant mRNA was co-introduced into T cells to normalize for the total amount of exogenous mRNA.

Flow Cytometry Analysis

Cells were harvested, washed once with cold FACS buffer (PBS with 1% FCS and 0.1% sodium azide) and incubated for 30 minutes at 4° C. in the dark with the respective Ab-conjugate at concentration recommended by the manufacturer. Cells were washed again with 4 ml FACS buffer, resuspended in 0.3 ml PBS with 0.1% sodium azide and subjected to flow cytometry using FACSCalibur or FACSAria II (Becton Dickinson, San Jose, Calif.). Data were analyzed by LSRII (BD) and FCSexpress (DeNovo Software, Los Angeles, Calif.).

T Cell Activation Assays

Chlorophenol red β-D galactopyranoside (CPRG) assay for B3Z T cell activation: Following cell- or antibody-mediated activation, growth medium was removed and 100 μl of lysis buffer (9 mM MgCl₂, 0.125% NP40, 0.3 mM CPRG in PBS) was added to each well. 1-24 hours post-lysis the optical density (O.D.) of each well was checked using ELISA reader (at 570 nm, with 630 nm as reference). For assaying antigen-specific human CD8 T cell response, cells were washed and co-cultured in complete medium with the respective melanoma target cells at an effector-to-target ratio of 1:1 for 24 hours. IFN-γ, TNF-α and GM-CSF secreted to the growth medium was monitored with commercial ELISA kits (R&D Systems Minneapolis, Minn.).

Luciferase Reporter Assay for NF-κB Activity

NF-κB activity was measured by transient transfection of the NF-κB-Luciferase reporter plasmid to various adherent cell lines together with the particular gene under study. Twelve to forty-eight hours post-transfection luciferase activity in the cell lysate was monitored by the Luciferase Assay Systems reagent (Promega), using Infinite M200 Pro microplate reader (Tecan, Männedorf, Switzerland).

Co-Culture Experiments

Seven hours post-electroporation T cells and melanoma target cells were co-cultured for 18 hours in triplicates at an effector-to-target ratio of 1:1 for B3Z and 3:1 for human CD8 T cells. For analysis of cell surface markers T cells were subjected to flow cytometry analysis using FACSCalibur (BD). For monitoring cytokine secretion growth medium was analyzed with commercial ELISA kits.

Cytotoxicity Assay

CD8 T cells and melanoma target cells were co-cultured as described above for the cytokine ELISA. After 18 hours of co-culture cells with growth medium were transferred to a FACS tube, centrifuged for 7 min at 1,500 RPM and the supernatant was assayed for target cell killing using a commercial kit for the lactate dehydrogenase (LDH) cell cytotoxicity assay (BioVision, Milpitas, Calif.).

Statistical Analysis

All results are presented as mean±SEM. ELISA results are shown as the mean of triplicates with standard error (SEM). Statistical significance was determined using multiple comparisons with a-parametric test, one-way ANOVA-Kruskal Wallis, in SPSS software or GraphPad Prism software.

### Example 1

### CD40-CARs Induce Stronger NFκB Activation Than 4-1BB-CARs

To create the new series of CARs we have employed the pGEM4Z/EGFP/A64 vector to produce a template DNA cassette for mRNA synthesis encoding a scFv derived from the anti-HLA-A2 mAb BB7.2, a Myc tag and the CD8a hinge at the ectodomain and the FcRγ intracellular T cell-activating domain (FIG. 1).

As the CD28 transmembrane domain was reported to support better surface expression than 4-1BB [44], we decided to similarly assess CD40, CD28 and 4-1BB. For this purpose we constructed four 2^{nd} generation CARs: 40(+)-γ and CD28(+)-γ, harboring the intact human CD40 or CD28 transmembrane and intracellular portion, respectively, and 40-γ and 4-1BB-γ, containing the transmembrane domain of CD28 and the intracellular domain of either CD40 or 4-1BB (FIG. 1). The two 3^{rd} generation CARs comprise the CD28 transmembrane and intracellular portion followed by either CD40 or 4-1BB intracellular domain.

Using the Myc tag it was confirmed by flow cytometry that all these CARs are properly expressed at the cell surface of K562 cells following electroporation of in-vitro transcribed mRNA (FIG. 2).

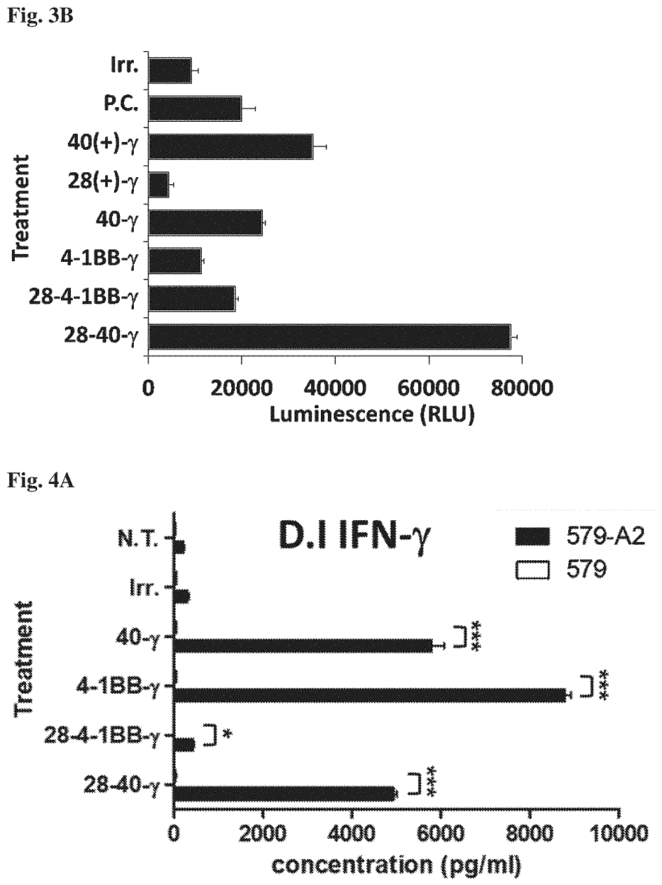

Proper cell surface expression and antigen-mediated T cell activation was assessed by employing the reporter mouse T cell hybridoma B3Z. We performed a co-culture experiment, assessing the ability of each of the six CARs to confer on B3Z transfectants the ability to respond to HLA-A2 on the 579-A2 melanoma cells in comparison with the parental 579 cells (FIG. 3A). While all CARs endowed B3Z cells with the anticipated antigenic specificity which, except for 40(+)-γ, was comparable in magnitude to that induced by the 2C11 anti-CD3 mAb, considerable level of antigen non-specific-response to the parental 579 cells was also evident. Interestingly, whereas the incorporation of the CD28 transmembrane domain in the 40-γ CAR did not translate into higher level of surface expression, it did result in a more robust response compared to 40(+)-γ.

We went on to evaluate the ability of the new CARs to activate the NF-κB signaling pathway, as judged by an NF-κB-Luciferase reporter assay in the HLA-A2(+) HEK293T cells. As can be seen in FIG. 3B, the 3^{rd} generation CAR 28-40-γ induced considerably stronger signaling than its 28-41BB-γ counterpart. Superiority of the 2^{nd} generation CARs 40(+)-γ and 40-γ over 41BB-γ was also observed, while 28(+)-γ was utterly inactive in this assay. Interestingly, in contrast to its weaker activation of the NFAT pathway in B3Z cells, expression of the 40(+)-γ construct resulted in stronger NF-κB activation than 40-γ.

### Example 2

### Antigen-Mediated Activation of Human CD8 T Cells

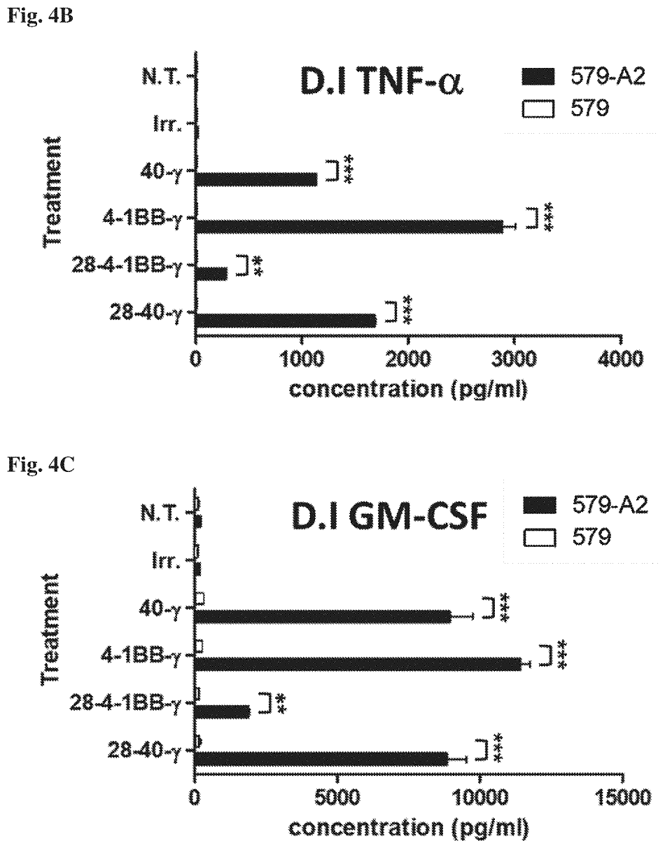

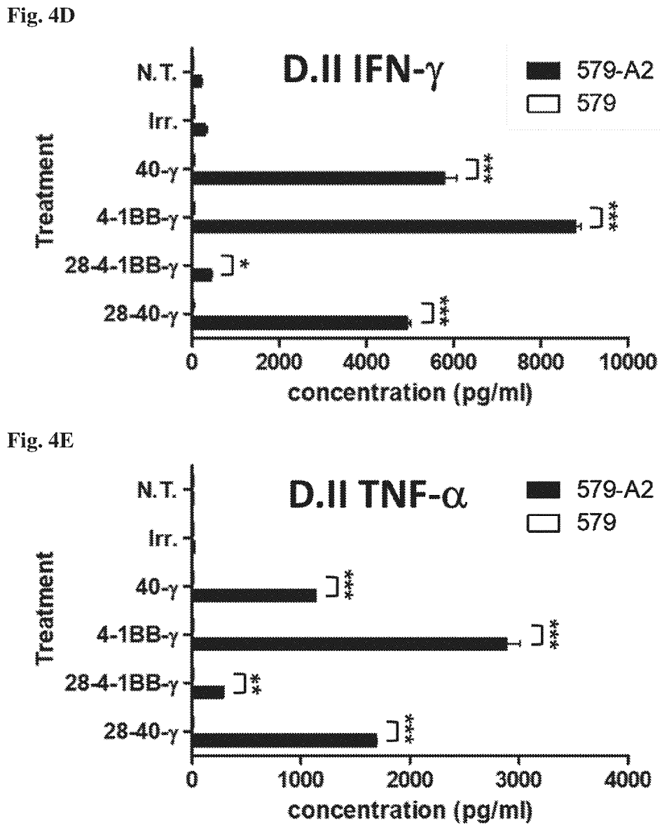

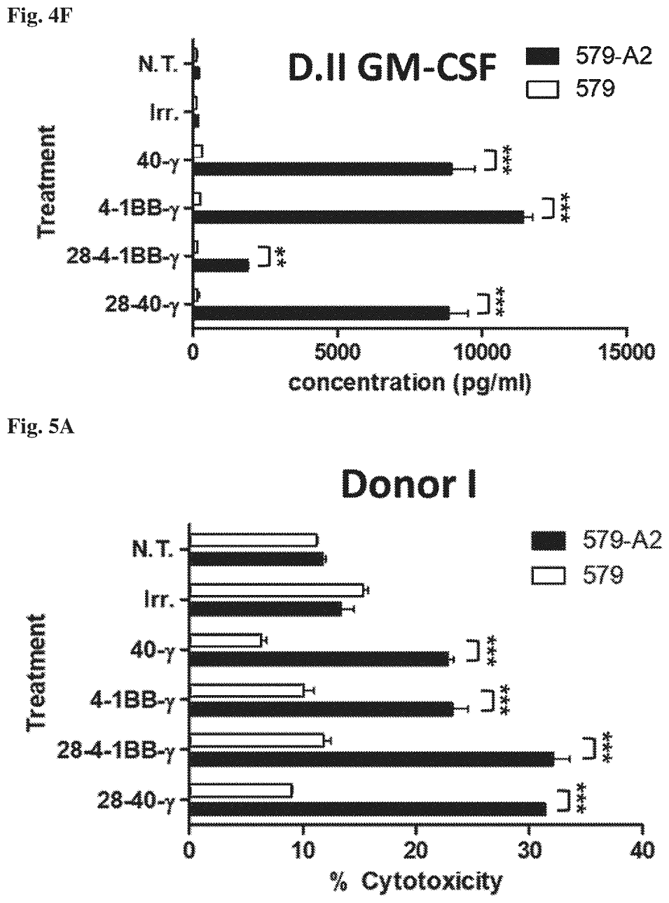

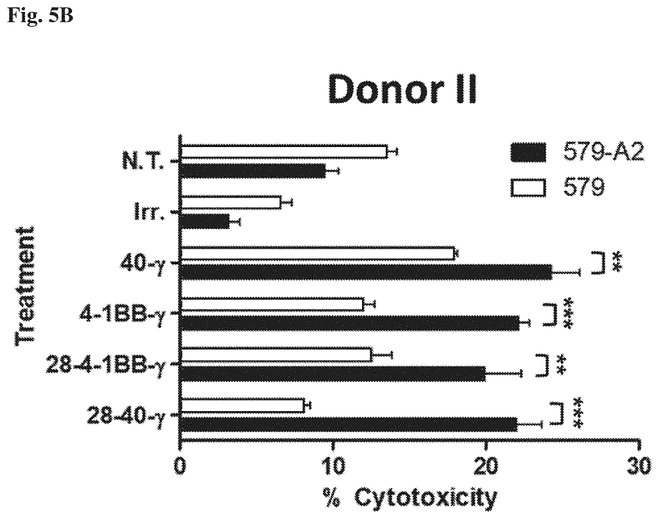

The functional properties conferred on human T cells were compared by the two pairs of 2nd and 3rd generation CARs following antigenic stimulus. To this end we purified CD8 T cells from peripheral blood samples of two HLA-A2(−) healthy donors. We transfected these cells with mRNA encoding each of the four CARs and irrelevant mRNA and then co-cultured transfectants with the 579-A2 and 579 melanoma cells. Following confirmation of amount and integrity of new mRNAs synthesized for the next series of experiments (not shown), mild surface expression following electroporation of CD8 T cells of the two donors was demonstrated (Not shown). Of note, unlike K562 cells, in which expression of 28-4-1BB-γ was comparable to the other CARs tested, 28-4-1BB-γ yielded relatively lower signal in the two unrelated CD8 T cell samples. In subsequent co-culture experiments, 579-A2, but not 579 cells induced upregulation of the CD25 and 4-1BB activation markers (Not shown). Similarly, only the antigen-positive 579-A2 cells induced secretion of the pro-inflammatory cytokines IFN-γ (FIG. 4A, D), TNF-α (FIG. 4B, E), and GM-CSF (FIG. 4C, F). Although differences emerged between the response patterns of CD8 T cells of the two donors, all CARs potentiated clear antigen-specific induction of the three cytokines with no or only minimal secretion in the absence of antigen. We now assessed antigen-mediated target cell killing (FIGS. 5A-B). Here, too, all four CARs in the two CD8 T cell samples could significantly mediate antigen-specific target cell killing, with no discernible differences between CD40 and 4-1BB in either CAR generation. Regarding the relatively low level of expression of 28-4-1BB-γ, it mediated effective cytotoxicity, comparable to that of 28-40-γ in CD8 T cells of the two donors, attesting to full competence of this mRNA.

### REFERENCES

1. Gross G, Waks T, Eshhar Z. Expression of immunoglobulin-T-cell receptor chimeric molecules as functional receptors with antibody-type specificity. Proc Natl Acad Sci U S A. 1989; 86:10024-8.

2. Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc Natl Acad Sci U S A. 1993; 90:720-4.

3. Gross G, Eshhar Z. Therapeutic Potential of T Cell Chimeric Antigen Receptors (CARs) in Cancer Treatment: Counteracting Off-Tumor Toxicities for Safe CAR T Cell Therapy. Annu Rev Pharmacol Toxicol [Internet]. 2016 [cited 2016 Jan. 12]; 56:59-83. Available from: http://www.ncbi.nlm.nih.gov/pubmed/26738472

4. Klebanoff C A, Rosenberg S A, Restifo N P. Prospects for gene-engineered T cell immunotherapy for solid cancers. Nat Med [Internet]. Nature Publishing Group, a division of Macmillan Publishers Limited. All Rights Reserved.; 2016 [cited 2016 Jan. 6]; 22:26-36. Available from: http://dx.doi.org/10.1038/nm.4015

5. Holzinger A, Barden M, Abken H. The growing world of CAR T cell trials: a systematic review. Cancer Immunol Immunother [Internet]. 2016 [cited 2016 Sep. 19]; 65:1433-50. Available from: http://www.ncbi.nlm.nih.gov/pubmed/27613725

6. Gajewski T F, Schreiber H, Fu Y-X. Innate and adaptive immune cells in the tumor microenvironment. Nat Immunol [Internet]. Nature Publishing Group, a division of Macmillan Publishers Limited. All Rights Reserved.; 2013 [cited 2014 Jul. 11]; 14:1014-22. Available from: http://dx.doi.org/10.1038/ni.2703

7. Crompton J G, Sukumar M, Restifo N P. Uncoupling T-cell expansion from effector differentiation in cell-based immunotherapy. Immunol Rev [Internet]. 2014; 257:264-76. Available from: http://dx.doi.org/10.1111/imr.12135

8. Stoiber S, Cadilha B L, Benmebarek M-R, Lesch S, Endres S, Kobold S, et al. Limitations in the Design of Chimeric Antigen Receptors for Cancer Therapy. Cells [Internet]. Multidisciplinary Digital Publishing Institute; 2019 [cited 2019 Jul. 10]; 8:472. Available from: https://www.mdpi.com/2073-4409/8/5/472

9. Long A H, Haso W M, Shern J F, Wanhainen K M, Murgai M, Ingaramo M, et al. 4-1BB costimulation ameliorates T cell exhaustion induced by tonic signaling of chimeric antigen receptors. Nat Med [Internet]. 2015 [cited 2019 Jul. 13]; 21:581-90. Available from: http://www.ncbi.nlm.nih.gov/pubmed/25939063

10. Zhao Z, Condomines M, van der Stegen S J C, Perna F, Kloss C C, Gunset G, et al. Structural Design of Engineered Costimulation Determines Tumor Rejection Kinetics and Persistence of CART Cells. Cancer Cell [Internet]. Elsevier; 2015 [cited 2015 Oct. 14]; 28:415-28. Available from: http://www.cell.com/article/S1535610815003359/fulltext

11. van der Stegen S J C, Hamieh M, Sadelain M. The pharmacology of second-generation chimeric antigen receptors. Nat Rev Drug Discov [Internet]. 2015 [cited 2015 Oct. 17]; 14:499-509. Available from: http://www.ncbi.nlm.nih.gov/pubmed/26129802

12. Acuto O, Michel F. CD28-mediated co-stimulation: a quantitative support for TCR signalling. Nat Rev Immunol [Internet]. 2003 [cited 2015 Sep. 9]; 3:939-51. Available from: http://dx.doi.org/10.1038/nri1248

13. Esensten J H, Helou Y A, Chopra G, Weiss A, Bluestone J A. CD28 Costimulation: From Mechanism to Therapy. Immunity [Internet]. 2016 [cited 2019 Jul. 11]; 44:973-88. Available from: https://linkinghub.elsevier.com/retrieve/pii/S1074761316301492

14. Deluca L S, Gommerman J L. Fine-tuning of dendritic cell biology by the TNF superfamily. Nat Rev Immunol [Internet]. Affiliation: University of Toronto, 1 King's College Circle, Toronto, ON M5S 1A8, Canada; 2012; 12:339-51. Available from: http://www.scopus.com/inward/record.url?eid=2-s2.0-84860244317&partnerID=40&md5=cab0ad17e7daf227008b8cfd96b01a7b

15. Lynch D H. The promise of 4-1BB (CD137)-mediated immunomodulation and the immunotherapy of cancer. Immunol Rev [Internet]. Bainbridge Biopharma Consulting, Bainbridge Island, Wash., USA. dhlynch@Gmail.com; 2008; 222:277-86. Available from: http://www.ncbi.nlm.nih.gov/entrez/query.fcgi?cmd=Retrieve&db=PubMed&dopt=Citation&list uids=18364008

16. Carpenito C, Milone M C, Hassan R, Simonet J C, Lakhal M, Suhoski M M, et al. Control of large, established tumor xenografts with genetically retargeted human T cells containing CD28 and CD137 domains [Internet]. Proc. Natl. Acad. Sci. U. S. A. Affiliation: Abramson Family Cancer Research Institute, University of Pennsylvania School of Medicine, Philadelphia, Pa. 19104, United States; Affiliation: Department of Pathology and Laboratory Medicine, University of Pennsylvania School of Medicine, Phil; 2009. p. 3360-5. Available from: http://www.scopus.com/inward/record.url?eid=2-s2.0-62549097817&partnerID=40&md5=80e3aa7f896aa764fe036f3ee7b05651

17. Milone M C, Fish J D, Carpenito C, Carroll R G, Binder G K, Teachey D, et al. Chimeric Receptors Containing CD137 Signal Transduction Domains Mediate Enhanced Survival of T Cells and Increased Antileukemic Efficacy In Vivo. Mol Ther [Internet]. 2009 [cited 2019 Jul. 12]; 17:1453-64. Available from: http://www.ncbi.nlm.nih.gov/pubmed/19384291

18. Kawalekar O U, O'Connor R S, Fraietta J A, Guo L, McGettigan S E, Posey A D, et al. Distinct Signaling of Coreceptors Regulates Specific Metabolism Pathways and Impacts Memory Development in CAR T Cells. Immunity [Internet]. 2016 [cited 2019 Jul. 12]; 44:712. Available from: http://www.ncbi.nlm.nih.gov/pubmed/28843072

19. Gomes-Silva D, Mukherjee M, Srinivasan M, Krenciute G, Dakhova O, Zheng Y, et al. Tonic 4-1BB Costimulation in Chimeric Antigen Receptors Impedes T Cell Survival and Is Vector-Dependent. Cell Rep [Internet]. 2017 [cited 2018 Jul. 24]; 21:17-26. Available from: http://linkinghub.elsevier.com/retrieve/pii/52211124717312767

20. Bourgeois C, Rocha B, Tanchot C. A role for CD40 expression on CD8+ T cells in the generation of CD8+ T cell memory. Science (80−) [Internet]. INSERM U345, Institut Necker, 156 Rue de Vaugirard, F-75730 Paris Cedex 15, France.; 2002; 297:2060-3. Available from: http://www.ncbi.nlm.nih.gov/entrez/query.fcgi?cmd=Retrieve&db=PubMed&dopt=Citation&list_uids=12242444

21. Munroe M E. Functional roles for T cell CD40 in infection and autoimmune disease: the role of CD40 in lymphocyte homeostasis. Semin Immunol [Internet]. Department of Microbiology, The University of Iowa, Iowa City, Iowa 52242, USA. melissa-munroe@uiowa.edu; 2009; 21:283-8. Available from: http://www.ncbi.nlm.nih.gov/entrez/query.fcgi?cmd=Retrieve&db=PubMed&dopt=Citation&list_uids=19539498

22. Soong RS, Song L, Trieu J, Lee SY, He L, Tsai Y C, et al. Direct T cell activation via CD40 ligand generates high avidity CD8+ T cells capable of breaking immunological tolerance for the control of tumors. PLoS One. Department of Pathology, Johns Hopkins Medical Institutions, Baltimore, Md., United States of America; Department of General Surgery, Chang Gung Memorial Hospital at Keelung, Keelung City, Taiwan; Chang Gung University, College of Medicine, Taoyuan,; 2014; 9:e93162.

23. Levin N, Pato A, Cafri G, Eisenberg G, Peretz T, Margalit A, et al. Spontaneous Activation of Antigen-presenting Cells by Genes Encoding Truncated Homo-Oligomerizing Derivatives of CD40. J Immunother [Internet]. 2016 [cited 2017 Feb. 26]; 40:39-50. Available from: http://content.wkhealth.com/linkback/openurl?sid=WKPTLP:landingpage&an=00002371-900000000-99550

24. Levin N, Weinstein-Marom H, Pato A, Itzhaki O, Besser M J, Eisenberg G, et al. Potent Activation of Human T Cells by mRNA Encoding Constitutively Active CD40. J Immunol [Internet]. 2018 [cited 2018 Nov. 30]; 201:2959-68. Available from: http://www.jimmunol.org/lookup/doi/10.4049/jimmuno1.1701725

25. Weinstein-Marom H, Levin N, Pato A, Shmuel N, Sharabi-Nov A, Peretz T, et al. Combined Expression of Genetic Adjuvants Via mRNA Electroporation Exerts Multiple Immunostimulatory Effects on Antitumor T Cells. J Immunother [Internet]. 2019 [cited 2019 Mar. 23]; 42:43-50. Available from: http://insights.ovid.com/crossref?an=00002371-201902000-00002

26. Foster A E, Mahendravada A, Shinners N P, Chang W-C, Crisostomo J, Lu A, et al. Regulated Expansion and Survival of Chimeric Antigen Receptor-Modified T Cells Using Small Molecule-Dependent Inducible MyD88/CD40. Mol Ther [Internet]. 2017 [cited 2018 Sep 22]; 25:2176-88. Available from: http://www.ncbi.nlm.nih.gov/pubmed/28697888

27. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer Discov [Internet]. 2017 [cited 2018 Aug. 17]; 7:1306-19. Available from: http://www.ncbi.nlm.nih.gov/pubmed/28801306

28. Collinson-Pautz M R, Chang W-C, Lu A, Khalil M, Crisostomo J W, Lin P-Y, et al. Constitutively active MyD88/CD40 costimulation enhances expansion and efficacy of chimeric antigen receptor T cells targeting hematological malignancies. Leukemia [Internet]. 2019 [cited 2019 Sep. 11]; 33:2195-207. Available from: http://www.nature.com/articles/s41375-019-0417-9

29. Narayanan P, Lapteva N, Seethammagari M, Levitt J M, Slawin K M, Spencer D M. A composite MyD88/CD40 switch synergistically activates mouse and human dendritic cells for enhanced antitumor efficacy. J Clin Invest [Internet]. Affiliation: Department of Pathology and Immunology, Baylor College of Medicine, One Baylor Plaza, Houston, Tex. 77030, United States; Affiliation: Diana Helis Henry Medical Research Foundation, New Orleans, La., United States; Affiliation: Scott Department; 2011; 121:1524-34. Available from: http://www.scopus.com/inward/record.url?eid=2-s2.0-79953308076&partnerID=40&md5=a3a418a0e2b41987d76761a6a9f5de2a

30. Xie P. TRAF molecules in cell signaling and in human diseases. J Mol Signal [Internet]. 2013 [cited 2019 Aug. 28]; 8:7. Available from: http://www.ncbi.nlm.nih.gov/pubmed/23758787

31. Wong JS, Wang X, Witte T, Nie L, Carvou N, Kern P, et al. Stalk region of beta-chain enhances the coreceptor function of CD8. J Immunol [Internet]. American Association of Immunologists; 2003 [cited 2019 Aug. 28]; 171:867-74. Available from: http://www.ncbi.nlm.nih.gov/pubmed/8283028

32. Chen X, Zaro J L, Shen W-C. Fusion protein linkers: property, design and functionality. Adv Drug Deliv Rev [Internet]. 2013 [cited 2016 Aug. 18]; 65:1357-69. Available from: http://www.pubmedcentral.nih.gov/articlerender.fcgi?artid=3726540&tool=pmcentrez&renderty pe=abstract

33. Reddy Chichili V P, Kumar V, Sivaraman J. Linkers in the structural biology of protein-protein interactions. Protein Sci [Internet]. 2013 [cited 2016 Aug. 18]; 22:153-67. Available from: http://www.pubmedcentral.nih.gov/articlerender.fcgi?artid=3588912&tool=pmcentrez&renderty pe=abstract

34. Whitlow M, Bell B A, Feng S L, Filpula D, Hardman K D, Hubert S L, et al. An improved linker for single-chain Fv with reduced aggregation and enhanced proteolytic stability. Protein Eng [Internet]. 1993 [cited 2018 Mar. 16]; 6:989-95. Available from: http://www.ncbi.nlm.nih.gov/pubmed/8309948

35. Matuskova M, Durinikov E. Retroviral Vectors in Gene Therapy. Adv Mol Retrovirology [Internet]. InTech; 2016 [cited 2019 Mar. 21]. Available from: http://www.intechopen.com/books/advances-in-molecular-retrovirology/retroviral-vectors-in-gene-therapy

36. Izsvák Z, Ivics Z. Sleeping Beauty Transposition: Biology and Applications for Molecular Therapy. Mol Ther [Internet]. 2004 [cited 2019 May 27]; 9:147-56. Available from: http://www.ncbi.nlm.nih.gov/pubmed/14759798

37. Miller A D, Law M F, Verma I M. Generation of helper-free amphotropic retroviruses that transduce a dominant-acting, methotrexate-resistant dihydrofolate reductase gene. Mol Cell Biol [Internet]. American Society for Microbiology (ASM); 1985 [cited 2020 Mar 7]; 5:431-7. Available from: http://www.ncbi.nlm.nih.gov/pubmed/2985952

38. Miller A D, Buttimore C. Redesign of retrovirus packaging cell lines to avoid recombination leading to helper virus production. Mol Cell Biol [Internet]. American Society for Microbiology (ASM); 1986 [cited 2019 Aug. 28]; 6:2895-902. Available from: http://www.ncbi.nlm.nih.gov/pubmed/3785217

39. Danos O, Mulligan R C. Safe and Efficient Generation of Recombinant Retroviruses with Amphotropic and Ecotropic Host Ranges [Internet]. Proc. Natl. Acad. Sci. U. S. A. National Academy of Sciences; 1988 [cited 2019 Aug. 28]. p. 6460-4. Available from: https://www.jstor.org/stable/32039

40. Bregni M, Magni M, Siena S, Di Nicola M, Bonadonna G, Gianni A. Human peripheral blood hematopoietic progenitors are optimal targets of retroviral-mediated gene transfer. Blood. 1992; 80:1418-22.

41. Xu L, Stahl S K, Dave H P, Schiffmann R, Correll P H, Kessler S, et al. Correction of the enzyme deficiency in hematopoietic cells of Gaucher patients using a clinically acceptable retroviral supernatant transduction protocol. Exp Hematol [Internet]. 1994 [cited 2020 Mar. 7]; 22:223-30. Available from: http://www.ncbi.nlm.nih.gov/pubmed/8299741

42. Hughes P F, Thacker J D, Hogge D, Sutherland H J, Thomas T E, Lansdorp P M, et al. Retroviral gene transfer to primitive normal and leukemic hematopoietic cells using clinically applicable procedures. J Clin Invest [Internet]. American Society for Clinical Investigation; 1992 [cited 2019 Aug. 28]; 89:1817-24. Available from: http://www.ncbi.nlm.nih.gov/pubmed/1601991

43. Boczkowski D, Nair S K, Nam J H, Lyerly H K, Gilboa E. Induction of tumor immunity and cytotoxic T lymphocyte responses using dendritic cells transfected with messenger RNA amplified from tumor cells. Cancer Res [Internet]. Center for Genetic and Cellular Therapies, Department of Surgery, Duke University Medical Center, Durham, N.C. 27710, USA.; 2000; 60:1028-34. Available from: http://www.ncbi.nlm.nih.gov/entrez/query.fcgi?cmd=Retrieve&db=PubMed&dopt=Citation&list_uids=10706120

44. Bishop DC, Xu N, Tse B, O'Brien T A, Gottlieb D J, Dolnikov A, et al. PiggyBac-Engineered T Cells Expressing CD19-Specific CARs that Lack IgG1 Fc Spacers Have Potent Activity against B-ALL Xenografts. Mol Ther [Internet]. 2018 [cited 2020 Apr. 24]; 26:1883-95. Available from: http://www.ncbi.nlm.nih.gov/pubmed/29861327

1. A nucleic acid molecule comprising a nucleotide sequence encoding an activating chimeric antigen receptor (aCAR) comprising:
(i) an extracellular binding domain;
(ii) a transmembrane domain;
(iii) an intracellular domain; and
(iv) a flexible hinge domain linking the extracellular binding domain and the transmembrane domain, said flexible hinge domain comprising a cysteine residue capable of forming a cysteine bridge,
wherein said intracellular domain is selected from:
(a) an intracellular domain comprising a first amino acid sequence comprising at least one signal transduction element derived from CD40, and a second amino acid sequence comprising at least one signal transduction element derived from an FcR gamma (γ) chain, CD3 zeta (ζ) chain, or CD3 eta (η) chain, and lacking a MyD88 polypeptide, 2A self-cleaving peptide or a dimerizing domain;
(b) an intracellular domain comprising a first amino acid sequence comprising at least one signal transduction element derived from CD40 and a third amino acid sequence comprising at least one signal transduction element derived from CD28 or 4-1BB; and
(c) an intracellular domain comprising a first amino acid sequence comprising at least one signal transduction element derived from CD40, a second amino acid sequence comprising at least one signal transduction element derived from an FcR gamma (γ) chain, CD3 zeta (ζ) chain, or CD3 eta (η) chain and a third amino acid sequence comprising at least one signal transduction element derived from CD28; and
(d) an intracellular domain comprising a first amino acid sequence comprising at least one signal transduction element derived from CD40 and lacking a MyD88 polypeptide or a dimerizing domain.

2. The nucleic acid molecule of claim 1, wherein said extracellular binding domain comprises (i) an antibody, derivative or fragment thereof, such as a humanized antibody; a human antibody; a functional fragment of an antibody; a single-domain antibody, such as a Nanobody; a recombinant antibody; and a single chain variable fragment (ScFv); (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer.

3. The nucleic acid molecule of claim 2, wherein said extracellular binding domain comprises an ScFv.

4. The nucleic acid molecule of claim 1, wherein said transmembrane domain is selected from the transmembrane domain of CD28, CD40, CD3-η TLR1, TLR2, TLR4, TLR5, TLR9, and Fc receptor.

5. The nucleic acid molecule of claim 4, wherein said transmembrane domain is the transmembrane domain of CD28.

6. The nucleic acid molecule of claim 1, wherein said first amino acid sequence is the complete intracellular domain of CD40.

7. The nucleic acid molecule of claim 1, wherein said at least one signal transduction element of said second amino acid sequence is derived from an FcRγ chain.

8. The nucleic acid molecule of claim 7, wherein said second amino acid sequence is the complete intracellular domain of an FcRγ chain.

9. The nucleic acid molecule of claim 1, wherein said third amino acid sequence is the complete intracellular domain of CD28.

10. The nucleic acid molecule of claim 1, wherein said flexible hinge comprises a polypeptide selected from a hinge region of CD8α, CD8β, a hinge region of a heavy chain of IgG, and a hinge region of a heavy chain of IgD.

11. The nucleic acid molecule of claim 10, wherein said flexible hinge domain is the hinge domain of CD8α.

12. The nucleic acid molecule of claim 1, wherein said extracellular binding domain comprises (i) an antibody, derivative or fragment thereof, such as a humanized antibody; a human antibody; a functional fragment of an antibody; a single-domain antibody, such as a Nanobody; a recombinant antibody; and a single chain variable fragment (ScFv); (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer; said transmembrane domain is selected from the transmembrane domain of CD28, CD3-η TLR1, TLR2, TLR4, TLR5, TLR9, and Fc receptor; said first amino acid sequence is the complete intracellular domain of CD40; said at least one signal transduction element of said second amino acid sequence is derived from an FcRγ chain; said third amino acid sequence is the complete intracellular domain of CD28; and said flexible hinge comprises a polypeptide selected from a hinge region of CD8α, CD8β, a hinge region of a heavy chain of IgG, and a hinge region of a heavy chain of IgD.

13. The nucleic acid molecule of claim 12, wherein said extracellular binding domain comprises an ScFv; said transmembrane domain is the transmembrane domain of CD28; said second amino acid sequence is the complete intracellular domain of an FcRγ chain; and said flexible hinge domain is the flexible hinge domain of CD8α.

14. The nucleic acid molecule of any one of claims 1 to 13, wherein said intracellular domain comprises a tandem arrangement of the complete intracellular domains of CD40-FcRγ.

15. The nucleic acid molecule of claim 14, wherein said aCAR comprises a tandem arrangement of ScFv-hinge region of CD8α-CD28 transmembrane domain-intracellular domain essentially consisting of a tandem arrangement of the complete intracellular domains of CD40-FcRγ.

16. The nucleic acid molecule of any one of claims 1 to 13, wherein said intracellular domain comprises a tandem arrangement of the complete intracellular domains of CD28-CD4O-FcRγ, wherein the intracellular domain of CD28 is optionally linked to the intracellular domain of CD40 via a linker.

17. The nucleic acid molecule of claim 16, wherein said intracellular domain comprises a tandem arrangement of the complete intracellular domains of CD28-linker-CD40-FcRγ.

18. The nucleic acid molecule of claim 16 or 17, wherein said aCAR comprises a tandem arrangement of ScFv-hinge region of CD8α-CD28 transmembrane domain-intracellular domain essentially consisting of a tandem arrangement of the complete intracellular domains of CD28-CD40-FcRγ, wherein the intracellular domain of CD28 is optionally linked to the intracellular domain of CD40 via a linker.

19. A composition comprising the nucleic acid molecule of any one of claims 1 to 18.

20. A vector comprising the nucleic acid molecule of any one of claims 1 to 18.

21. A mammalian T cell comprising the nucleic acid molecule of any one of claims 1 to 18, or the DNA vector of claim 20.

22. The mammalian T cell of claim 21, which is a CD4⁺ helper T cell or regulatory T cell (Treg).

23. The mammalian T cell of claim 21, which is a CD8⁺ effector T cell.

24. The mammalian T cell of any one of claims 21 to 23, expressing on its surface said aCAR.

25. The mammalian Treg of any one of claims 21 to 24, which is a human T cell.

26. A method of preparing allogeneic or autologous aCAR T cells, the method comprising contacting T cells with the nucleic acid molecule of any one of claims 1 to 18; or a vector of claim 20, thereby preparing allogeneic or autologous aCAR T cells.
